(19) United States
(12) Patent Application Publication
(10) Pub. No.: US 2026/0263732 A1
(43) Pub. Date: Sep. 10, 2026

(54) BILATERAL SIMULATION DEVICE AND A METHOD THEREOF

(71) Applicant: Ava M. Hart, Middletown, CT (US)

(72) Inventor: Ava M. Hart, Middletown, CT (US)

(73) Assignee: Ava M. Hart, Middletown, CT (US)

(21) Appl. No.: 19/071,654

(22) Filed: Mar. 5, 2025

(51) Int. Cl. A61M 21/02; A61M 21/00; G16H 20/70

(52) U.S. Cl. CPC A61M 21/02; G16H 20/70; A61M 2021/0022; A61M 2021/0027; A61M 2021/0044; A61M 2205/3303; A61M 2205/3306; A61M 2205/3375; A61M 2205/502; A61M 2205/583

(57) ABSTRACT

A bilateral simulation device is disclosed. The bilateral simulation device comprises a body, a plurality of simulation elements configured to provide at least one of tactile, or auditory simulations at varying intensities and having a plurality of modes. Further, at least one interface module configured to receive a first set of information. Further, at least one processor is configured to determine a second set of information for the one or more users using an artificial intelligence (AI) module; determine at least one mode from the plurality of modes of simulation; generate one or more command signals based on to the determined at least one mode; and send the generated one or more command signals to the plurality of simulation elements and the at least one display module for providing at least one of the visual simulation, the tactile simulation and/or the auditory simulation.

### FIELD OF THE DISCLOSURE

The invention relates to a simulation device. More particularly, the invention relates to a bilateral simulation device and a method thereof.

### BACKGROUND OF THE DISCLOSURE

The subject matter discussed in this background section should not be assumed to be prior art merely as a result of its mention herein. Similarly, any problems mentioned in this background section or associated with the subject matter of this background section should not be assumed to have been previously recognized in the prior art. The subject matter as disclosed in this background section merely represents different approaches related to providing bilateral simulations to multiple users at a same time, wherein such device and method themselves may also correspond to implementations of the claimed technology and invention.

In today's fast-paced and high-stress world, individuals increasingly face various physiological and emotional challenges that affect their overall well-being. Such issues can manifest in many forms, including anxiety, stress, depression, fatigue, and emotional distress, often leading to negative impacts on personal health, productivity, and quality of life. As a result, there has been a growing need for solutions that help individuals to manage or mitigate these issues in a supportive and an effective manner. Traditional approaches to addressing emotional and physiological concerns often involve therapy, medication, or physical interventions. While these methods can be effective, they typically involve one-on-one interactions or rely on passive treatment approaches that may not fully engage the individual or address the complexity of human emotional responses. Additionally, some individuals may not have access to such therapies, or they may be reluctant to seek professional help due to stigma or privacy concerns.

Simulation devices such as virtual reality (VR) systems, have shown promise in offering immersive environments that can help individuals to manage stress, anxiety, and other emotional challenges. The simulation devices can create controlled, calming experiences, or expose users to scenarios that allow them to practice emotional regulation and coping mechanisms. However, existing systems are often designed for use by a single user at a time, which limits their potential for fostering shared experiences or supporting social interaction i.e., key components of emotional well-being. Moreover, these existing systems may fail to adapt to the specific physiological responses or emotional states of users, offering a generalized experience rather than one that is personalized to each user's unique needs.

In group or therapeutic settings such as support groups, family counselling, or team-building exercises, there is often a need for simulation devices that can address the physiological and emotional issues of multiple users simultaneously. These environments can promote collective healing and support, enabling users to engage in shared emotional experiences, receive peer support, and improve their interpersonal relationships. However, most existing solutions do not provide the necessary customization and real-time interaction for managing the diverse emotional states and physiological conditions of multiple participants.

Prior art, for various aspects contained there within, relevant to this disclosure includes US Pat. Publication No. 11865268 B2 to Neta, U.S. Pat. Publication No. 20210213239 A1 to Stacy, and US Pat. Publication No. 10328235 B2 to Kelly. These prior arts describe systems that utilize bilateral simulation (BLS) for therapeutic and physiological benefits. However, this is not an ideal solution for providing a device that enables group or multi-person therapy sessions by coordinating bilateral simulation for multiple individuals. These systems typically focus on single-user applications or fail to synchronize the therapeutic experience for multiple participants. As a result, these systems lack the capability to provide a shared, adaptive, and interactive environment that could address the unique physiological and emotional needs of each individual within a group setting. This limitation hinders the potential for more effective group-based therapeutic sessions, where each participant's experience could be personalized and aligned with the overall group dynamics.

In particular, reference '268 to Neta discloses about systems and methods for Remote Eye Movement Desensitization and Reprocessing (EMDR) therapy that include performing, at therapist and client computing platforms, steps of receiving an EMDR message indicating a position a bilateral simulation (BLS) element, wherein the BLS element is at least one of a visual, an audio, and a tactile element, and wherein the indicated position is a screen position of a visual element, or a right or left position of an audio or tactile element; and responsively presenting, on the respective computing platform the BLS element at the indicated position; and performing on the EMDR server steps of: establishing connections with the therapist and client computing platforms; and broadcasting simultaneously, over the first and second connections to both the therapist and client computing platforms, the EMDR message. However, this system is limited to one-on-one interactions and does not incorporate real-time, personalized adjustments based on the client's physiological or emotional conditions. Further, the device lacks the ability to provide bilateral simulation device at both hemispheres of the brain by using visual, auditory and tactile methods by identifying residual tension or discomfort using additional simulation. Further, the device lacks the use of a cognitive interweave to guide processing of bilateral simulation by questioning beliefs or providing contextual insights. Further, the device does not disclose a reassessment phase to reassess distress, ensures emotional stability with grounding, encourages reflection, and plans follow-up support and does not disclose monitoring of progress and addresses any residual or emerging issues to support long-term integration. Further, the device does not disclose a feature of receiving a rating from the client based on current feelings such as stress level, to improve the bilateral simulation methods.

Reference '239 to Stacy discloses about bilateral simulation devices, systems, and methods. In one embodiment, a bilateral simulation device can include a housing, a tactile stimulator coupled to the housing, a communications module configured to receive wireless communication from a remote device; and a controller module configured to independently and selectively control at least one tactile setting of the tactile stimulator based on wireless communication received from the remote device. However, the device primarily focuses on tactile simulation for a single user and lacks the ability to integrate multiple users or adapt the therapy based on real-time emotional or physiological feedback, limiting its overall therapeutic scope. Further, the device lacks the ability to provide bilateral simulation device at both hemispheres of the brain by using visual, auditory and tactile methods by identifying residual tension or discomfort using additional simulation. Further, the device lacks the use of a cognitive interweave to guide processing of bilateral simulation by questioning beliefs or providing contextual insights. Further, the device does not disclose a reassessment phase to reassess distress, ensures emotional stability with grounding, encourages reflection, and plans follow-up support and does not disclose monitoring of progress and addresses any residual or emerging issues to support long-term integration. Further, the device does not disclose a feature of receiving the rating from client based on current feelings such as stress level, to improve the bilateral simulation methods.

Reference '235 to Kelly discloses about a system and method that provides controlled bilateral simulation to a patient in response to physiological feedback to initiate and/or accelerate the onset of sleep and to maintain the sleep state after the onset of sleep. The system induces bilateral simulation through simulation modules that are coupled to opposite lateral sides of the patient's body. The simulation modules stimulate the patient's body bilaterally through a sequence of simulations that alternate from one side of the patient's body to the other with a pause between each successive simulation. The system controls the duration of the simulations, the intensity of the simulations and the period of time between successive simulations in response to feedback from physiological sensors. The sensors are coupled to the patient and provide physiological information which initiates or terminates the bilateral simulations, and/or modifies the characteristics of the simulations. However, this system is specifically focused on sleep-related applications and lacks the ability to address a wider range of therapeutic needs or engage multiple users simultaneously. Further, the device lacks the ability to provide bilateral simulation device at both hemispheres of the brain by using visual, auditory and tactile methods by identifying residual tension or discomfort using additional simulation. Further, the device lacks the use of a cognitive interweave to guide processing of bilateral simulation by questioning beliefs or providing contextual insights. Further, the device does not disclose a reassessment phase to reassess distress, ensures emotional stability with grounding, encourages reflection, and plans follow-up support and does not disclose monitoring of progress and addresses any residual or emerging issues to support long-term integration. Further, the device does not disclose a feature of receiving the rating from client based on current feelings such as stress level, to improve the bilateral simulation methods.

Given the deficiencies of the prior art, the need remains for a device that can provide coordinated bilateral simulation for multiple users simultaneously, adapting the therapy to each individual's physiological and emotional needs in real-time. Unlike existing solutions, which focus on single-user applications or limited remote control, this device would facilitate group-based therapy, offering personalized and dynamic therapeutic experiences. It would integrate real-time feedback, such as physiological responses or emotional conditions, to ensure that each participant receives tailored simulation, fostering more effective and immersive therapeutic sessions.

### SUMMARY OF THE DISCLOSURE

According to embodiments illustrated herein, a novel, simple and interactive device and method thereof.

In some embodiments, the invention discloses about a bilateral simulation device designed to offer personalized therapeutic experiences for multiple users. The bilateral simulation device provides a range of simulations-visual, tactile, and auditory—while adjusting settings such as intensity and pause duration to enhance the therapy's effectiveness. Furthermore, the bilateral simulation device allows for the modification of simulation characteristics in response to each user's emotional or physiological state, ensuring a highly adaptive and personalized therapy session. The present invention offers several key advantages in providing personalized and adaptive therapeutic experiences through bilateral simulation. The present invention enables dynamic, real-time customization of tactile, auditory, and visual simulations based on each user's psychological and emotional conditions, such as trauma or anxiety. By using an AI-powered processor, the bilateral simulation device tailors the simulation modes (e.g., duration, intensity) to suit individual therapeutic goals, ensuring more effective and personalized therapy. Additionally, the integration of physiological feedback through sensors allows the system to continuously adjust based on real-time bodily responses, enhancing the therapy's responsiveness. This approach fosters a more immersive, adaptive, and interactive environment for therapy, making it a more effective solution for addressing complex emotional and physiological conditions.

In some embodiments, a bilateral simulation device is disclosed, the bilateral simulation device comprises a body, a plurality of simulation elements coupled to the body via one or more extensible connections. Further, the plurality of simulation elements are configured to provide at least one of tactile, or auditory simulations at varying intensities and the plurality of simulation elements having a plurality of modes. Further, the bilateral simulation device comprises at least one interface module communicatively coupled to the plurality of simulation elements and configured to provide a visual simulation. Further, the at least one interface module is configured to receive a first set of information from the one or more users, where the first set of information comprises at least one of phycological or emotional conditions and historical data of each of the one or more users. Further, the bilateral simulation device comprises at least one processor communicatively coupled to the plurality of simulation elements, the at least one interface module, and a memory having a plurality of instructions which when executed by the at least one processor to determine a second set of information for the one or more users based at least on the first set of information using an artificial intelligence (AI) module, where the second set of information comprises at least one of a therapeutic goal of each of the one or more users or real time bodily sensations; determine at least one mode from the plurality of modes for each of the one or more users based at least on the determined second set of information using the AI module, where the plurality of modes comprises duration of simulation, type of simulation, and duration between two subsequent simulations provided by the plurality of simulation elements and the at least one interface module; generate one or more command signals corresponding to the determined at least one mode from the plurality of modes for each of the one or more users; and send the generated one or more command signals to at least one of the plurality of simulation elements for providing at least one of the auditory simulation or the tactile simulation to the one or more users, or the at least one interface module for providing the visual simulation to the one or more users.

In some embodiments, the plurality of simulation elements comprises a plurality of vibrating elements configured to provide the tactile simulation and one or more speaker elements configured to provide the auditory simulation.

In some embodiments, the phycological or emotional conditions comprises at least one of trauma, anxiety, or phobias. Further, the type of simulations comprises the tactile simulation, the auditory simulation and the visual simulation.

In some embodiments, the therapeutic goal corresponds to areas of improvement for each of the one or more users, wherein the therapeutic goal comprises at least one of behavior change, coping skills, self-esteem, or communication.

In some embodiments, the at least one processor using the AI module is configured to guide, via the at least one interface module, each of the one or more users to think about traumatic events; monitor, via one or more sensors communicatively coupled to the at least one processor, one or more actions of each of the one or more users, wherein the one or more actions correspond to at least one of auditory or visual actions of each of the one or more users; and determine the real time bodily sensations based at least on the monitored one or more actions of each of the one or more users.

In some embodiments, the the one or more sensors comprises at least one of a microphone configured to capture the auditory actions and an image capturing device configured to capture visual actions.

In some embodiments, the at least one interface module corresponds to a digital interface module, and comprises a plurality of blinking indicators configured to indicate the visual simulation.

In some embodiments, the at least one processor, using the AI module, is further configured to receive a first rating corresponding to collective distress using a subjective units of distress scale (SUDS) from 0 to 10 from each of the one or more users, via the at least one interface module, wherein the first rating corresponds to a rating received from each of the one or more users prior to providing the simulation; receive a second rating corresponding to collective distress using the SUDS from 0 to 10 from each of the one or more users, via the at least one interface module, wherein the second rating corresponds to a rating received from each of the one or more users after providing the simulation; compare the first rating with the second rating of each of the one or more users; and determine whether the second rating is higher than the first rating based at least on the comparison.

In some embodiments, upon determining that the second rating is higher than the first rating, the at least one processor is configured to determine that an improvement in the collective distress has occurred and to provide follow-up sessions corresponding to the simulation to the one or more users.

In some embodiments, wherein upon determining that the second rating is lower than or equal to the first rating, the at least one processor is configured to determine that no improvement in the collective distress has occurred, wherein the at least one processor is configured to update the determined at least one mode from the plurality of modes for each of the one or more users upon determining that no improvement in the collective distress.

In some embodiments, the at least one processor is configured to train the AI module based at least on the historical data, the first rating, the second rating and the determined at least one mode for each of the one or more users.

In some embodiments, a method is disclosed. The method comprises steps of receiving, via at least one interface module communicatively coupled to a plurality of simulation elements, a first set of information from the one or more users, wherein the first set of information comprises at least one of phycological or emotional conditions and historical data of each of the one or more users, wherein the plurality of simulation elements provides at least one of tactile or auditory simulation at varying intensities, and wherein the at least one interface module is configured to provide a visual simulation; determining, via at least one processor communicatively coupled to a plurality of simulation elements, the at least one interface module, and a memory having a plurality of instructions, a second set of information for the one or more users based at least on the first set of information using an artificial intelligence (AI) module, wherein the second set of information comprises at least one of a therapeutic goal of each of the one or more users or real time bodily sensations; determining, via the at least one processor, at least one mode from the plurality of modes for each of the one or more users based at least on the determined second set of information using the AI module, wherein the plurality of modes comprises duration of simulation, a type of simulation, and duration between two subsequent simulations provided by the plurality of simulation elements and the at least one interface module; generating, via the at least one processor, one or more command signals based on the determined at least one mode from the plurality of modes for each of the one or more users; and sending, via the at least one processor, the generated one or more command signals to at least one of the plurality of simulation elements for providing at least one of the auditory simulation or the tactile simulation to the one or more users, or the at least one interface module for providing the visual simulation to the one or more users.

### BRIEF DESCRIPTION OF THE DRAWINGS

While the specification concludes with claims particularly pointing out and distinctly claiming particular embodiments of the present disclosure, various embodiments of the present disclosure can be more readily understood and appreciated from the following descriptions of various embodiments of the present disclosure when read in conjunction with the accompanying drawings, in which:

FIG. 1 illustrates a block diagram of a bilateral simulation system, according to an example embodiment of the present invention;

FIGS. 2A-2B illustrate an overview of one or more users interacting with a bilateral simulation device, according to an example embodiment of the present invention;

FIG. 3 illustrates an exemplary table showing distress levels, personal history, and specific therapeutic needs of the one or more users, according to an example embodiment of the present invention; and

FIG. 4 illustrates a flowchart showing a method for bilateral simulation, according to an example embodiment of the present invention.

### DETAILED DESCRIPTION

Reference will now be made in detail to specific embodiments or features, examples of which are illustrated in the accompanying drawings. Wherever possible, corresponding or similar reference numbers will be used throughout the drawings to refer to the same or corresponding parts. Moreover, references to various elements described herein, are made collectively or individually when there may be more than one element of the same type. However, such references are merely exemplary in nature. It may be noted that any reference to elements in the singular may also be construed to relate to the plural and vice-versa without limiting the scope of the disclosure to the exact number or type of such elements unless set forth explicitly in the appended claims.

Some embodiments of this disclosure, illustrating all its features, will now be discussed in detail. The words “comprising,” “having,” “containing,” and “including,” and other forms thereof, are intended to be equivalent in meaning and be open-ended in that an item or items following any one of these words is not meant to be an exhaustive listing of such item or items, or meant to be limited to only the listed item or items.

It must also be noted that as used herein and in the appended claims, the singular forms “a,” “an,” and “the” include plural references unless the context dictates otherwise. Although any systems and methods similar or equivalent to those described herein can be used in the practice or testing of embodiments of the present disclosure, the preferred systems, and methods are now described.

Embodiments of the present disclosure will be described more fully hereinafter with reference to the accompanying drawings in which like numerals represent like elements throughout the several figures, and in which example embodiments are shown. Embodiments of the present disclosure may, however, be embodied in alternative forms and should not be construed as being limited to the embodiments set forth herein. The examples set forth herein are non-limiting examples and are merely examples among other possible examples.

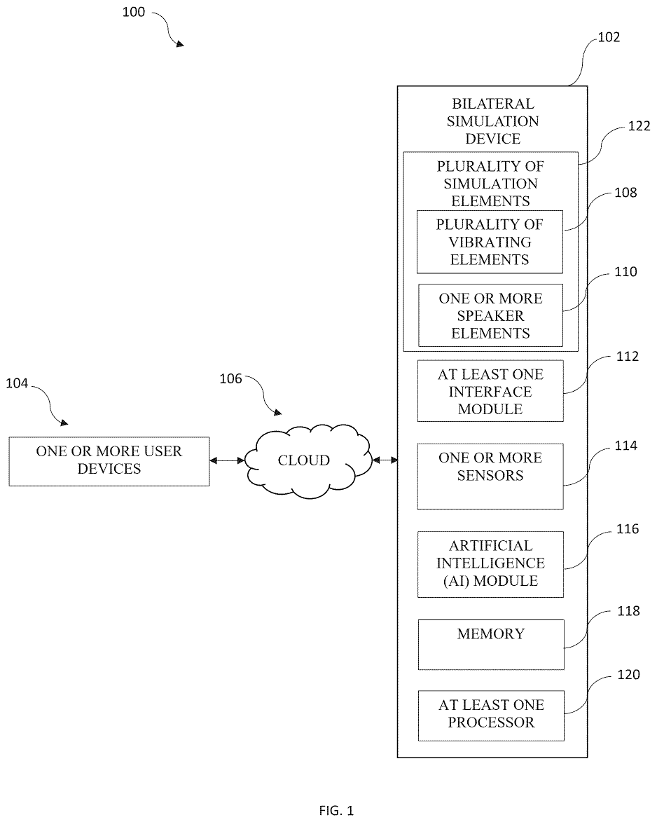

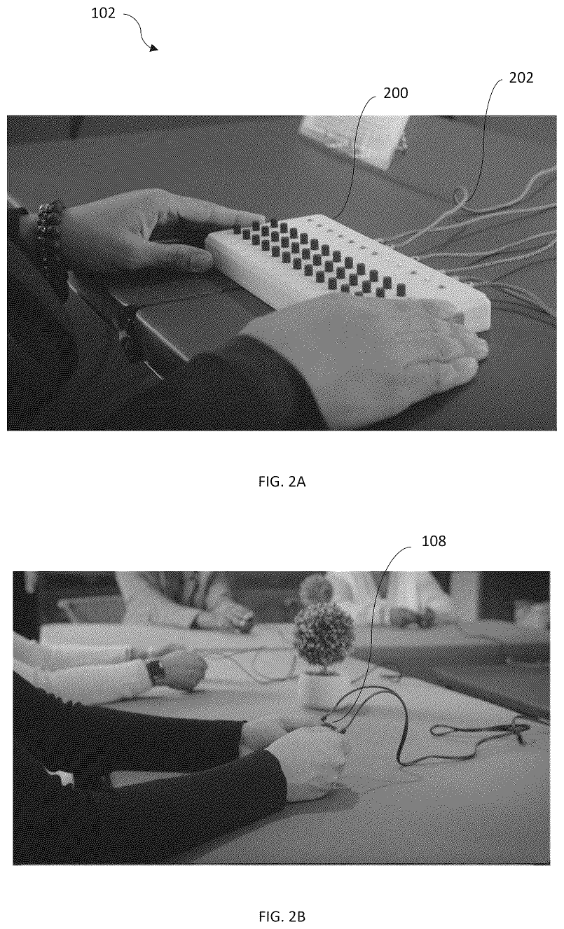

FIG. 1 illustrates a block diagram of a bilateral simulation system 100 showing a communication between a bilateral simulation device 102 and one or more user devices 104, in accordance with an example embodiment of the present invention. FIGS. 2A-2B illustrate an overview of one or more users interacting with the bilateral simulation device 102, according to an example embodiment of the present invention.

In some embodiments, the bilateral simulation system 100 comprises the bilateral simulation device 102, the one or more user devices 104, and a cloud 106. In some embodiments, the bilateral simulation device 102 is configured to provide bilateral simulation to the one or more users at a same time. The bilateral simulation device 102 disclosed herein is designed to provide simultaneous, bilateral simulation to the one or more users, that enables immersive and interactive experiences. The bilateral simulation device 102 employs elongated, flexible one or more extensible connections 202, to facilitate portability and accessibility, ensuring ease of use in handheld configurations. The bilateral simulation device 102 incorporates advanced bilateral simulation capabilities, allowing synchronized or alternating simulation to both sides of a user's body or among the one or more users, thereby enhancing engagement and therapeutic outcomes. The bilateral simulation device 102 ensures precise delivery of simulation signals, which includes tactile, visual, auditory, or electrical feedback, tailored to meet the needs of diverse use cases.

In some embodiments, the bilateral simulation device 102 not only addresses accessibility but also introduces a novel approach for facilitating simultaneous multi-user interaction, making the bilateral simulation device 102 particularly suitable for applications such as therapy, training, gaming, or rehabilitation. In some embodiments, the bilateral simulation device 102 comprises a body 200, a plurality of simulation elements 122 comprising a plurality of vibrating elements 108, one or more speaker elements 110, at least one interface module 112, one or more sensors 114, at least one AI (artificial intelligence) module 116, at least one processor 120, and a memory 118.

In some embodiments, the body 200 of the bilateral simulation device 102 serves as a central framework that houses and supports the essential components of the bilateral simulation device 102. The body 200 is designed to be sturdy yet lightweight, ensuring both durability and ease of use for one or more users. The body 200 may be constructed from high-quality materials, offering a balance between strength and flexibility. The shape and structure of the body 200 is optimized for comfort, ensuring that the bilateral simulation device 102 be easily adjusted for use by the one or more users in various therapeutic environments. The design of the body 200 allows for seamless integration with other device components, ensuring stability while enabling effective bilateral simulation.

In some embodiments, the bilateral simulation device 102 further comprises the plurality of simulation elements 122. In some embodiments, the plurality of simulation elements 122 is coupled to the body 200 via the one or more extensible connections 202. In some embodiments, the one or more extensible connections 202 are designed to be flexible and adjustable, allowing the plurality of simulation elements 122 to be positioned or moved to accommodate the one or more users and provide tailored simulation. The extensible nature of the one or more extensible connections 202 ensures that the bilateral simulation device 102 may be easily modified for various therapeutic settings, allowing the one or more users to adjust the placement and configuration of the plurality of simulation elements 122 for optimal comfort and effectiveness. Additionally, the one or more extensible connections 202 may facilitate the distribution of tactile simulation, auditory simulation, or visual simulation in a way that enhances the therapeutic experience, promoting better engagement and responsiveness during bilateral simulation session.

In some embodiments, the plurality of simulation elements 122 are accessible to each of the one or more users to provide at least one of tactile, or auditory simulations at varying intensities and having a plurality of modes of simulation. In some embodiments, the plurality of simulation elements 122 comprises the plurality of vibrating elements 108 and the one or more speaker elements 110. In some embodiments, the plurality of vibrating elements 108 are designed to provide tactile simulation to the user. The plurality of vibrating elements 108 are strategically positioned on the bilateral simulation device 102 to create a balanced and immersive experience. Each vibrating element of the plurality of vibrating elements 108 may be independently controlled to vary intensity and frequency, allowing for a customized therapeutic effect.

In some embodiments, the plurality of vibrating elements 108 are coupled to the body 200 of the bilateral simulation device 102 via the extensible connections 202, ensuring flexibility in placement and alignment of the plurality of vibrating elements 108. The flexibility in the placement of the plurality of vibrating elements 108 enables the bilateral simulation device 102 to adapt to the specific needs of the one or more users, providing targeted simulation to areas of body that require attention. The use of the plurality of vibrating elements 108 enhances the overall effectiveness of the bilateral simulation by providing consistent, synchronized tactile feedback, which may be adjusted in real-time based on the one or more user's emotional or physiological responses.

In some embodiments, the plurality of vibrating elements 108 may generate tactile simulation through small motors (not shown) or piezoelectric actuators (not shown) that create rapid oscillations or vibrations. The motors or the piezoelectric actuators are activated by electrical signals, with intensity and pause duration of the vibrations adjustable based on therapeutic needs. The vibrations are transmitted to the one or more user's body through a contact surface (gripped by hand of the one or more users as shown in FIG. 2B) of the plurality of vibrating elements 108, providing targeted sensory feedback. Each vibrating element of the plurality of vibrating elements 108 may be independently controlled, allowing for precise modulation of the simulation.

Further, the one or more speaker elements 110 is designed to provide auditory simulation as part of the bilateral simulation to the one or more users. The one or more speaker elements 110 is configured to function by converting electrical signals into sound waves, producing varying frequencies and volumes of sound. The one or more speaker elements 110 is strategically positioned to deliver auditory simulation in a manner that is both effective and comfortable for the one or more users. The output of the one or more speaker elements 110 may be adjusted to different intensities and patterns, allowing to be synchronized with other forms of simulation (e.g., tactile or visual) to create a multi-sensory therapeutic experience. By providing auditory feedback, the one or more speaker elements 110 may contribute to overall sensory simulation, enhancing the ability of the bilateral simulation device 102 to address psychological and emotional conditions in a personalized and immersive manner.

The adjustment of the intensities and patterns will be discussed in the following paragraphs. In one alternative embodiments, the plurality of simulation elements 122 may also be configured to provide other forms of simulation. The other forms of simulation may comprise visual simulation. The one or more speaker elements 110 provides alternating tones or beeps delivered through headphones or speakers. These are effective for the one or more users who prefer listening over visual focus.

In some embodiments, the bilateral simulation device 102 further comprises the at least one interface module 112. In some embodiments, the interface module 112 corresponds to a digital interface module 112. In some embodiments, the at least one interface module 112 may communicatively be coupled to the plurality of simulation elements 122 and configured to provide a visual simulation. The visual simulation comprises a moving light or object (e.g., LEDs on the at least one interface module 112 moving left-right that is tracked by each of the one or more users. These are effective for the one or more users who are comfortable with visual engagement and focus.

In some embodiments, the at least one interface module 112 is configured to receive a first set of information from the one or more users, where the first set of information comprises at least one of phycological or emotional conditions and historical data of each of the one or more users. In some embodiments, the phycological or emotional conditions comprises at least one of trauma, anxiety, or phobias. Further, the historical data may comprise at least medical history of each of the one or more users.

Further, the bilateral simulation device 102 may comprise the at least one processor 120. The at least one processor 120 is communicatively coupled to the plurality of vibrating elements 108, the at least one interface module 112, and the memory 118 having a plurality of instructions. The at least one processor 120 is configured to determine a second set of information for the one or more users based at least on the first set of information using the artificial intelligence (AI) module 116. In some embodiments, the second set of information comprises at least one of a therapeutic goal of each of the one or more users or real time bodily sensations. The therapeutic goal is determined from the first set of information and corresponds to areas of improvement for the one or more users. The therapeutic goal comprises at least one of behavior change, coping skills, self-esteem, or communication.

In some embodiments, the AI module 116 is responsible for personalizing the therapeutic experience by processing and analysing both real-time and the historical data from the one or more users. Initially, the at least one processor 120 via the AI module 116 receives the first set of information, including the one or more user's psychological or emotional conditions (such as trauma or anxiety, heart rate or body temperature). Further the at least one processor 120 is configured to determine real time bodily sensations of each of the one or more users using the one or more sensors 114. The one or more sensor are communicatively coupled to the at least one processor 120.

In some embodiments, the at least one processor 120 in order to determine the real time bodily sensations, is configured to guide each of the one or more users to think about traumatic events using the at the at least one interface module 112. The at least one interface module 112 is configured to display one or more instructions to each of the on more users to guide or instruct the one or more users to think about the traumatic events. Further, the one or more sensors 114 are configured to monitor one or more actions of each of the one or more users. In some embodiments, the one or more actions correspond to at least one of auditory or visual actions of each of the one or more users. Further, the at least one processor 120 using the AI module 116, is configured to determine the real time bodily sensations based at least on the monitored one or more actions of each of the one or more users.

In some embodiments, the one or more sensors 114 comprises at least one microphone and an image capturing device. In some embodiments, the at least one microphone is configured to capture the auditory actions of each of the one or more users and send to the at least one processor 120. Further, the image capturing device is configured to capture the visual actions of each of the one or more users and send to the at least one processor 120. In some embodiments, based on the determination of the visual actions and the auditory actions, the at least one processor 120 is configured to determine the real time bodily sensations of each of the one or more users.

In some embodiments, the at least one processor 120 using the AI module 116 is further configured to determine therapeutic goals (e.g., behaviour change or emotional regulation). This enables the AI module 116 to continuously adjust the simulation parameters—such as intensity, duration, or frequency—during therapy to ensure an optimal, adaptive experience for each user. Further, in some embodiments, the at least one processor 120 is further configured to determine at least one mode from the plurality of modes of simulation for each of the one or more users based at least on the determined the second set of information using the AI module 116. In some embodiments, the plurality of modes comprises duration of simulation, a type of simulation, and duration between two subsequent simulations provided by the plurality of simulation elements and the at least one interface module 112. In some embodiments, the at least one processor 120 using the plurality of simulation elements 122 and the at least one interface module 112, is configured to provide alternating simulations (left-right signals).

In some embodiments, the bilateral simulation device 102 further comprises a pause duration control unit (not shown) and a vibrational control unit (not shown). In some embodiments, the pause duration control unit is configured to control pause duration and the vibrational control unit configured to control the intensity, of the visual simulation, the tactile simulation and the auditory simulation for each of the one or more users based at least on the determined mode. Further, the at least one interface module 112 comprises a plurality of blinking indicators (not shown) configured to indicate the visual simulation and a counter configured to determine a number of activation cycles of the visual simulation.

Further, the at least one processor 120 is configured to generate one or more command signals corresponding to the determined at least one mode from the plurality of modes for each of the one or more users, for providing the simulation as shown in FIG. 2B. Further, the at least one processor 120 is configured to send the generated one or more command signals corresponding to the determined at least one mode to the plurality of simulation elements 122 and the at least one interface module 112 for providing at least one of the visual simulation, the tactile simulation or the auditory simulation.

In some embodiments, the at least one processor 120 using the AI module 116, is further configured to receive a first rating corresponding to collective distress using a subjective unit of distress scale (SUDS) from 0 to 10, via the at least one interface module 112. In some embodiments, the first rating corresponds to a rating received from each of the one or more users prior to providing the simulation. Further, the at least one processor 120 using the AI module 116 is configured to receive a second rating corresponding to collective distress using the SUDS from 0 to 10, via the at least one interface module 112. In some embodiments, the second rating corresponds to a rating received from each of the one or more users after providing the simulation.

Further, the at least one processor 120 using the AI module 116 is configured to compare the first rating with the second rating of each of the one or more users. Further, the at least one processor 120 using the AI module 116 is configured to determine whether the second rating is higher than the first rating based at least on the comparison. Further, upon determining that the second rating is higher than the first rating, the at least one processor 120 is configured to determine that an improvement in the collective distress has occurred and to provide follow-up sessions corresponding to the simulation to the one or more users.

In some embodiments, upon determining that the second rating is lower than or equal to the first rating, the at least one processor is configured to determine that no improvement in the collective distress has occurred. Further, the at least one processor 120 is configured to either update the determined at least one mode from the plurality of modes for each of the one or more users upon determining that no improvement in the collective distress.

In some embodiments, the bilateral simulation device 102 is further communicatively connected to the one or more user devices 104 via the cloud 106. In some embodiments, the one or more user devices 104 corresponds to computing device equipped by each of the one or more users. In one example embodiments, the one or more user devices 104 comprises at least one of a mobile phone, computers, desktops, laptops etc. The connection between the bilateral simulation device 102 and the one or more user devices 104 enables seamless communication between the bilateral simulation device 102 and the one or more user devices 104, allowing the bilateral simulation device 102 to receive and transmit information in real-time.

In some embodiments, the cloud 106 is configured to facilitate the synchronization of data such as users' emotional and physiological feedback, between the bilateral simulation device 102 and the one or more user devices 104. In some embodiments, the connectivity between the bilateral simulation device 102 and the one or more user devices 104 allows each of the one or more users to adjust the plurality of modes for providing the bilateral simulation, ensuring personalized and effective therapy. Additionally, the cloud 106 enables remote monitoring, control, and management of bilateral simulation sessions, ensuring that each user's experience may be tailored and adapted as needed during the bilateral simulation session.

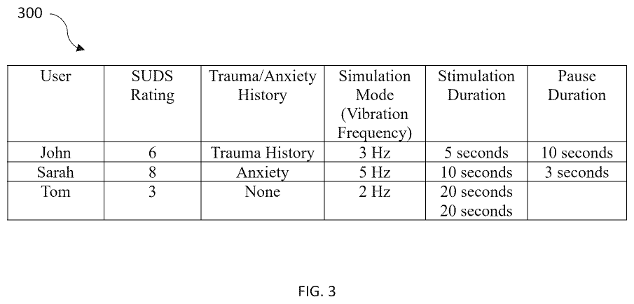

FIG. 3 illustrates an exemplary table 300 showing distress levels, personal history, and specific therapeutic needs of the one or more users, according to an example embodiment of the present invention.

In one example embodiment, three individuals John, Sarah, and Tom participated in a group bilateral simulation session using the bilateral simulation device 102 designed to address trauma, anxiety, and stress. In some embodiments, each participant are screened by checking if they are suitable for receiving bilateral simulation based on their historical data. Further, each participant is educated using the at least one interface module 112, by explaining the purpose of the bilateral simulation session, how the bilateral simulation device 102 works, and what to expect during bilateral simulation session.

Further, the at least one processor 120 using the at least one interface module 112 is configured to ensure participants feel safe and secure in the therapy space by teaching grounding strategies, such as breathing exercises, for the one or more users to use during and after. The teaching of grounding strategies, such as breathing exercises is done by displaying text over the at least one interface module 112. Each user is sat in front of the at least one interface module 112. Each of the one or more users are arranged in a circular or semi-circular layout to maintain focus on the group dynamic while accommodating individual comfort. Further, the plurality of simulation elements 122, including the plurality of vibrating elements 108 and the one or more speaker elements 110, are strategically placed for optimal engagement.

In one example embodiments, John has a history of trauma and is feeling moderate distress as indicated by a SUDS rating of 6. The at least one processor 120, using the AI module 116, analyzes John's historical data and current distress level. Based on this, the AI module 116 determines that he needs a moderate intensity and shorter duration of simulation to help him focus on the traumatic events while ensuring he doesn't become overwhelmed. Further, John's simulation mode is set to a vibration frequency of 3 Hz with a 5-second duration for each stimulus and a 10-second pause between stimuli.

Further, Sarah, has a relatively high anxiety level but no significant trauma history. She rates her distress level as 8 on the SUDS scale. The AI module 116 determines that she requires a higher intensity of simulation to address her anxiety, with a longer duration for each of the bilateral simulation session and shorter breaks between stimuli. Her settings are adjusted to 5 Hz vibrations and 10-second simulation intervals with a 3-second pause between stimuli. Further, Tom has low levels of anxiety and no significant trauma. Tom's SUDS rating is 3. Since Tom less distressed, the AI module 116 adjusts his simulation mode to a mild intensity and longer pauses between stimuli. His settings include 2 Hz vibration frequency with a 20-second pause between each simulation for a more relaxed, less intense bilateral simulation session.

In some embodiments, throughout the bilateral simulation session, the at least one processor 120 continually monitors their physiological responses and adjusts the simulation parameters accordingly, ensuring that each user receives a tailored and responsive therapeutic experience. In some embodiments, the at least one processor 120 using the image capturing device, checks in with the one or more users regularly to guide the bilateral simulation session and to address emerging thoughts or emotions. Further, the at least one processor 120 is also configured to pause stimulation to check in with the one or more users, asking for brief feedback or observations. The at least one processor 120 is configured to adjust the intensity of lights produced from the at least one interface module 112 and sounds or vibrations from the plurality of simulation elements 122 so that the bilateral simulation is neither too faint (ineffective) nor too strong (overwhelming).

In some embodiments, the bilateral simulation comprises cognitive interweave phase. In the cognitive interweave phase, the at least one processor 120 is configured to determine, using the image capturing device, the microphone and the AI module 116, if the one or more users appear stuck or unable to process distress by observing one or more auditory actions and the visual actions. Based on which the bilateral simulation device 102 is configured to use the cognitive interweave to guide bilateral simulation. In one Example embodiments, the at least one interface module 112 may ask questions that include: questioning the belief: “What does this memory say about you today?”, providing context: “That event is in the past; what can you take from it now?” and challenging cognitive distortions: “Would you say this is entirely your fault, or are there other factors involved?”.

Further, the bilateral simulation device 102 using the at least one interface module 112 may guide the one or more users to focus on a positive belief related to the trauma (e.g., “We survived,” “We are resilient”) and help the one or more users to connect this belief to their body and emotional state. The bilateral simulation help the individuals confront their emotional issues by providing targeted, bilateral simulation based on real-time distress levels and personal histories (as shown in the FIG. 3). In some embodiments, the bilateral simulation device 102 encourages the one or more users to share experiences if comfortable, by using either the at least one interface module 112 or the one or more users device 104 for fostering a sense of connection.

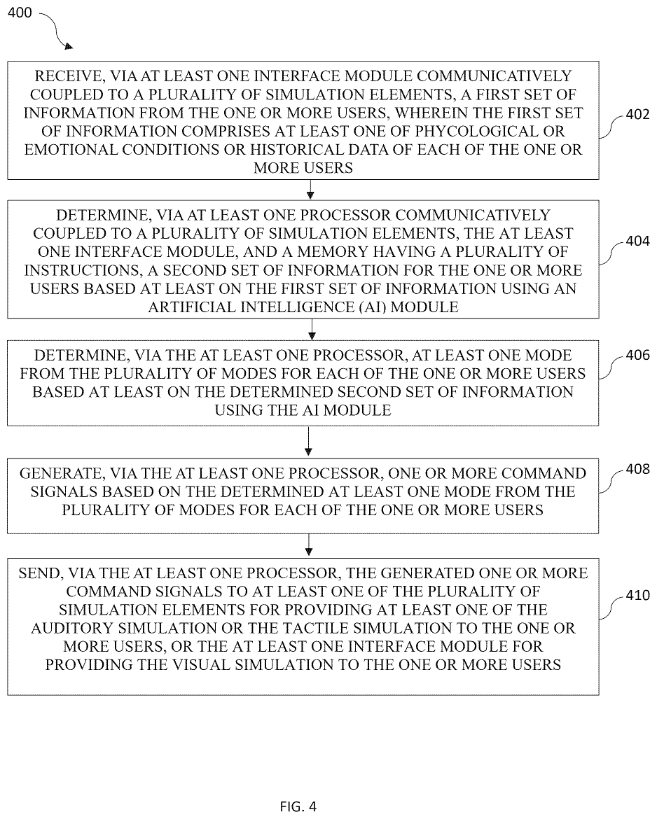

FIG. 4 illustrates a flowchart 400 showing a method for bilateral simulation, according to an example embodiment of the present invention.

At step 402, the at least one processor 120 is configured to receive, via at least one interface module 112 configured to provide the visual simulation, communicatively coupled to the plurality of simulation elements 122 configured to provide at least one of tactile or auditory simulation, the first set of information from the one or more users, where the first set of information comprises at least one of phycological or emotional conditions and historical data. In some embodiments, the plurality of simulation elements comprises a plurality of vibrating elements 108 and the one or more speaker elements 110.

Further, at step 404, the at least one processor 120 is configured to determine the second set of information for the one or more users based at least on the first set of information using an artificial intelligence (AI) module 116. In some embodiments, the second set of information comprises at least one of a therapeutic goal of each of the one or more users or real time bodily sensations. The therapeutic goal corresponds to areas of improvement for the one or more users, where the therapeutic goal comprises at least one of behavior change, coping skills, self-esteem, or communication.

In some embodiments, the at least one processor 120 is configured to guide each of the one or more users to think about traumatic events. Further, the at least one processor 120 is configured to monitor one or more actions of each of the one or more users, wherein the one or more actions correspond to at least one of auditory or visual actions of each of the one or more users. Further, the at least one processor 120 is configured to determine the real time bodily sensations based at least on the monitored one or more actions of each of the one or more users. In some embodiments, the one or more sensors 114 comprises at least one of the microphone configured to receive the auditory actions and the image capturing device configured to receive visual actions.

Further, at step 406, the at least one processor 120 is configured to determine at least one mode from the plurality of modes of simulation for each of the one or more users based at least on the determined the second set of information using the AI module 116. Further, the plurality of modes comprises duration of simulation and duration between two subsequent simulations provided by the plurality of simulation elements and/or the at least one interface module. In some embodiments, the at least one processor 120 further is configured to control pause duration of the visual simulation, the tactile simulation and/or the auditory simulation for each of the one or more users based at least on the determined mode using the pause duration control unit.

Further, the at least one processor 120 is configured to control the intensity of the visual simulation, the tactile simulation or the auditory simulation for each of the one or more users based at least on the determined mode. At step 408, the at least one processor 120 is configured to generate one or more command signals corresponding to the determined at least one mode from the plurality of modes for each of the one or more users, for providing the simulation. Further, the at least one processor 120 is configured to indicate via the plurality of blinking indicators of the at least one interface module, the visual simulation and determine via a counter of the at least one interface module, a number of activation cycles of the visual simulation.

Further, at step 410, the at least one processor 120 is configured to send the generated one or more command signals corresponding to the determined at least one mode to the plurality of simulation elements 122 for providing at least one of the auditory simulation or the tactile simulation to the one or more users and to the at least one interface module 112 for providing at least one of the visual simulation.

In some embodiments, the at least one processor 120 using the AI module 116, is further configured to receive a first rating corresponding to collective distress using a subjective unit of distress scale (SUDS) from 0 to 10, via the at least one interface module 112. In some embodiments, the first rating corresponds to a rating received from each of the one or more users prior to providing the simulation. Further, the at least one processor 120 using the AI module 116 is configured to receive a second rating corresponding to collective distress using the SUDS from 0 to 10, via the at least one interface module 112. In some embodiments, the second rating corresponds to a rating received from each of the one or more users after providing the simulation.

Further, the at least one processor 120 using the AI module 116 is configured to compare the first rating with the second rating of each of the one or more users. Further, the at least one processor 120 using the AI module 116 is configured to determine whether the second rating is higher than the first rating based at least on the comparison. Further, upon determining that the second rating is higher than the first rating, the at least one processor 120 is configured to determine that an improvement in the collective distress has occurred and to provide follow-up sessions corresponding to the simulation to the one or more users.

In some embodiments, upon determining that the second rating is lower than or equal to the first rating, the at least one processor is configured to determine that no improvement in the collective distress has occurred. Further, the at least one processor 120 is configured to either update the determined at least one mode from the plurality of modes for each of the one or more users upon determining that no improvement in the collective distress.

The bilateral simulation device 102 provides a highly personalized therapeutic experience by adapting to each user's emotional and physiological state in real-time. Using an AI-driven module, it analyzes distress levels, therapeutic goals, and historical data to adjust simulation parameters such as intensity, duration, and frequency, ensuring optimal therapy for each individual. This customization is particularly beneficial in group bilateral simulation sessions, where multiple users can receive tailored experiences simultaneously, making it an effective solution for collective emotional healing while maintaining individual needs. Additionally, the bilateral simulation device 102 offers real-time feedback and adjustment based on the user's responses, ensuring that the therapy remains aligned with their emotional state throughout the bilateral simulation session.

Furthermore, the bilateral simulation device 102 integrates a multi-sensory approach, combining tactile, auditory, and visual stimuli to enhance emotional processing and healing. The real-time monitoring and adjustment of these sensory elements allow the device to provide a controlled, safe, and effective therapeutic environment. By offering a holistic sensory experience and maintaining personalized settings, the bilateral simulation device 102 fosters improved emotional processing, allowing users to confront and process trauma or anxiety more effectively. This leads to a therapeutic process that is both dynamic and responsive, promoting long-term emotional resilience and growth.

While there is shown and described herein certain specific structures embodying various embodiments of the invention, it will be manifest to those skilled in the art that various modifications and rearrangements of the parts may be made without departing from the spirit and scope of the underlying inventive concept and that the same is not limited to the particular forms herein shown and described except insofar as indicated by the scope of the appended claims.

### LIST OF ELEMENTS

100—Bilateral simulation system
102—Bilateral simulation device
104—One or more user devices
106—Cloud
108—Plurality of vibrating elements
110—One or more speaker elements
112—At least one interface module
114—One or more sensors
116—AI module
118—Memory
120—At least one processor
122—Plurality of simulation elements
200—Body
202—One or more extensible connections
300—Table
400—Method
402—Step
404—Step
406—Step
408—Step
410—Step

What is claimed is:

1. A bilateral simulation device comprising:
a body;
a plurality of simulation elements coupled to the body via one or more extensible connections, wherein the plurality of simulation elements are configured to provide at least one of tactile or auditory simulation at varying intensities, the plurality of simulation elements having a plurality of modes;
at least one interface module communicatively coupled to the plurality of simulation elements and configured to provide a visual simulation, wherein the at least one interface module is configured to receive a first set of information from the one or more users, wherein the first set of information comprises at least one of phycological or emotional conditions or historical data of each of the one or more users;
at least one processor communicatively coupled to the plurality of simulation elements, the at least one interface module, and a memory having a plurality of instructions which when executed by the at least one processor to:
determine a second set of information for each of the one or more users based at least on the first set of information using an artificial intelligence (AI) module, wherein the second set of information comprises at least one of a therapeutic goal of each of the one or more users or real time bodily sensations;
determine at least one mode from the plurality of modes for each of the one or more users based at least on the determined second set of information using the AI module, wherein the plurality of modes comprises duration of simulation, a type of simulation, and duration between two subsequent simulations provided by the plurality of simulation elements and the at least one interface module;
generate one or more command signals based on the determined at least one mode from the plurality of modes for each of the one or more users; and
send the generated one or more command signals to at least one of:
the plurality of simulation elements for providing at least one of the auditory simulation or the tactile simulation to the one or more users, or
the at least one interface module for providing the visual simulation to the one or more users.

2. The bilateral simulation device of claim 1, wherein the plurality of simulation elements comprises:
a plurality of vibrating elements configured to provide the tactile simulation; and
one or more speaker elements configured to provide the auditory simulation.

3. The bilateral simulation device of claim 1, wherein the phycological or emotional conditions comprises at least one of trauma, anxiety, or phobias, wherein the type of simulation comprises the at least one of tactile simulation, the auditory simulation, or the visual simulation.

4. The bilateral simulation device of claim 1, wherein the therapeutic goal corresponds to areas of improvement for each of the one or more users, wherein the therapeutic goal comprises at least one of behavior change, coping skills, self-esteem, or communication.

5. The bilateral simulation device of claim 1, wherein the at least one processor, using the AI module, is configured to:
guide, via the at least one interface module, each of the one or more users to think about traumatic events;
monitor, via one or more sensors communicatively coupled to the at least one processor, one or more actions of each of the one or more users, wherein the one or more actions correspond to at least one of auditory or visual actions of each of the one or more users; and
determine the real time bodily sensations based at least on the monitored one or more actions of each of the one or more users.

6. The bilateral simulation device of claim 5, wherein the one or more sensors comprises at least one of a microphone configured to capture the auditory actions and an image capturing device configured to capture the visual actions.

7. The bilateral simulation device of claim 1, wherein the at least one interface module corresponds to a digital interface module, and comprising a plurality of blinking indicators configured to indicate the visual simulation.

8. The bilateral simulation device of claim 1, wherein the at least one processor, using the AI module, is further configured to:
receive a first rating corresponding to collective distress using a subjective units of distress scale (SUDS) from 0 to 10 from each of the one or more users, via the at least one interface module, wherein the first rating corresponds to a rating received from each of the one or more users prior to providing simulation;
receive a second rating corresponding to collective distress using the SUDS from 0 to 10 from each of the one or more users, via the at least one interface module, wherein the second rating corresponds to a rating received from each of the one or more users after providing the simulation; and
compare the first rating with the second rating of each of the one or more users; and
determine whether the second rating is higher than the first rating based at least on the comparison.

9. The bilateral simulation device of claim 8, wherein upon determining that the second rating is higher than the first rating, the at least one processor is configured to determine that an improvement in the collective distress has occurred and to provide follow-up sessions corresponding to the simulation to the one or more users.

10. The bilateral simulation device of claim 8, wherein upon determining that the second rating is lower than or equal to the first rating, the at least one processor is configured to determine that no improvement in the collective distress has occurred.

11. The bilateral simulation device of claim 10, wherein the at least one processor is configured to:
update the determined at least one mode from the plurality of modes for each of the one or more users upon determining that no improvement in the collective distress.

12. The bilateral simulation device of claim 8, wherein the at least one processor is configured to train the AI module based at least on the historical data, the first rating, the second rating, and the determined at least one mode for each of the one or more users.

13. A method comprising:
receiving, via at least one interface module communicatively coupled to a plurality of simulation elements, a first set of information from the one or more users, wherein the first set of information comprises at least one of phycological or emotional conditions or historical data of each of the one or more users, wherein the plurality of simulation elements provides at least one of tactile or auditory simulation at varying intensities, and wherein the at least one interface module is configured to provide a visual simulation;
determining, via at least one processor communicatively coupled to the plurality of simulation elements, the at least one interface module, and a memory having a plurality of instructions, a second set of information for the one or more users based at least on the first set of information using an artificial intelligence (AI) module, wherein the second set of information comprises at least one of a therapeutic goal of each of the one or more users or real time bodily sensations;
determining, via the at least one processor, at least one mode from the plurality of modes for each of the one or more users based at least on the determined second set of information using the AI module, wherein the plurality of modes comprises duration of simulation, a type of simulation, and duration between two subsequent simulations provided by the plurality of simulation elements and the at least one interface module;
generating, via the at least one processor, one or more command signals based on the determined at least one mode from the plurality of modes for each of the one or more users; and
sending, via the at least one processor, the generated one or more command signals to at least one of:
the plurality of simulation elements for providing at least one of the auditory simulation or the tactile simulation to the one or more users, or
the at least one interface module for providing the visual simulation to the one or more users.

14. The method of claim 13, wherein the plurality of simulation elements comprises:
a plurality of vibrating elements configured to provide the tactile simulation; and
one or more speaker elements configured to provide the auditory simulation.

15. The method of claim 13, wherein the phycological or emotional conditions comprises at least one of trauma, anxiety, or phobias, wherein the type of simulation comprises the at least one of tactile simulation, the auditory simulation, or the visual simulation; and wherein the therapeutic goal corresponds to areas of improvement for each of the one or more users, wherein the therapeutic goal comprises at least one of behavior change, coping skills, self-esteem, or communication; and wherein the at least one interface module corresponds to a digital interface module.

16. The method of claim 11 further comprising:
guiding, via the at least one processor using the AI module, each of the one or more users to think about traumatic events via the at least one interface module;
monitoring, via one or more sensors communicatively coupled to the at least one processor, one or more actions of each of the one or more users, wherein the one or more actions correspond to at least one of auditory or visual actions of each of the one or more users, wherein the one or more sensors comprises at least one of a microphone for capturing the auditory actions and an image capturing device for capturing visual actions; and
determining, via the at least one processor using the AI module, the real time bodily sensations based at least on the monitored one or more actions of each of the one or more users.

17. The method of claim 13, further comprising:
receiving, via the at least one processor using the AI module, a first rating corresponding to collective distress using a subjective units of distress scale (SUDS) from 0 to 10 from each of the one or more users, via the at least one interface module, wherein the first rating corresponds to a rating received from each of the one or more users prior to providing simulation;
receiving, via the at least one processor using the AI module, a second rating corresponding to collective distress using the SUDS from 0 to 10 from each of the one or more users, via the at least one interface module, wherein the second rating corresponds to a rating received from each of the one or more users after providing the simulation; and
comparing, via the at least one processor using the AI module, the first rating with the second rating of each of the one or more users; and
determining, via the at least one processor using the AI module, whether the second rating is higher than the first rating based at least on the comparison.

18. The method of claim 17, wherein upon determining that the second rating is higher than the first rating, the at least one processor is configured to determine that an improvement in the collective distress has occurred and to provide follow-up sessions corresponding to the simulation to the one or more users, and wherein upon determining that the second rating is lower than or equal to the first rating, the at least one processor is configured to determine that no improvement in the collective distress has occurred.

19. The method of claim 17, further comprising:
updating, via the at least one processor, the determined at least one mode from the plurality of modes for each of the one or more users upon determining that no improvement in the collective distress.

20. The method of claim 17, further comprising:
training, via the at least one processor, the AI module based at least on the historical data, the first rating, the second rating, and the determined at least one mode for each of the one or more users.
